(19) United States
(12) Patent Application Publication
(10) Pub. No.: US 2015/0329442 A1
(43) Pub. Date: Nov. 19, 2015

(54) RECOVERY OPTIONS FOR n-BUTANE DEHYDROGENATION FOR ON-PURPOSE BUTADIENE PRODUCTION

(71) Applicant: UOP LLC, Des Plaines, IL (US)

(72) Inventor: John J. Senetar, Naperville, IL (US)

(73) Assignee: UOP LLC, Des Plaines, IL (US)

(21) Appl. No.: 14/278,040

(22) Filed: May 15, 2014

(51) Int. Cl. C07C 5/327; C07C 5/48

(52) U.S. Cl. CPC C07C 5/327; C07C 5/48

(57) ABSTRACT

A process for the production of butadiene is presented. The process converts a butane stream through dehydrogenation to process stream having butenes. The butenes are treated and separated from the process stream to generate an olefin rich stream. The olefin rich stream is passed to an oxydehydrogenation reactor for conversion to butadienes. The process utilizes a cold-box separation process for efficiently separating the process stream generated through dehydrogenation to separate light gases from the C4 hydrocarbon stream.

### FIELD OF THE INVENTION

The present invention relates to a process for the production of butadiene. In particular, this is a process for the integration of a butadiene production process into a petrochemical plant and utilizes an efficient method of separation of C4s from light gases.

### BACKGROUND

The use of plastics and rubbers are widespread in today's world. The production of these plastics and rubbers are from the polymerization of monomers which are generally produced from petroleum. The monomers are generated by the breakdown of larger molecules to smaller molecules which can be modified. The monomers are then reacted to generate larger molecules comprising chains of the monomers. An important example of these monomers is light olefins, including ethylene and propylene, which represent a large portion of the worldwide demand in the petrochemical industry. Light olefins, and other monomers, are used in the production of numerous chemical products via polymerization, oligomerization, alkylation and other well-known chemical reactions. Producing large quantities of light olefin material in an economical manner, therefore, is a focus in the petrochemical industry. These monomers are essential building blocks for the modern petrochemical and chemical industries. The main source for these materials in present day refining is the steam cracking of petroleum feeds.

Another important monomer is butadiene. Butadiene is a basic chemical component for the production of a range of synthetic rubbers and polymers, as well as the production of precursor chemicals for the production of other polymers. Examples include homopolymerized products such as polybutadiene rubber (PBR), or copolymerized butadiene with other monomers, such as styrene and acrylonitrile. Butadiene is also used in the production of resins such as acrylonitrile butadiene styrene.

Butadiene is typically recovered as a byproduct from the cracking process, wherein the cracking process produces light olefins such as ethylene and propylene. With the increase in demand for rubbers and polymers having the desired properties of these rubbers, an aim to improving butadiene yields from materials in a petrochemical plant will improve the plant economics

### SUMMARY

A first embodiment of the invention is a process for the dehydrogenation of n-butane comprising passing a first stream comprising n-butane to a dehydrogenation reactor to generate a second stream comprising n-butane and butenes; passing the second stream to a drying unit to generate a dried second stream passing the dried second stream to a cold-box condensing unit to generate a third stream comprising liquid C4 hydrocarbons and a fourth stream comprising lighter gases; passing the third stream to an olefin separation process to generate a fifth stream rich in olefins and a sixth stream rich in paraffins; and passing the fifth stream to an oxydehydrogenation reactor to generate a product stream comprising butadienes. An embodiment of the invention is one, any or all of prior embodiments in this paragraph up through the first embodiment in this paragraph wherein the butadienes in the product stream comprising 1,3 butadiene. An embodiment of the invention is one, any or all of prior embodiments in this paragraph up through the first embodiment in this paragraph further comprising passing the second stream with reduced water content to a chloride treater to generate a treated second stream. An embodiment of the invention is one, any or all of prior embodiments in this paragraph up through the first embodiment in this paragraph further wherein the olefin separation comprises passing the third stream to a selective olefin absorption process to generate the fifth stream comprising olefins and the sixth stream comprising paraffins. An embodiment of the invention is one, any or all of prior embodiments in this paragraph up through the first embodiment in this paragraph wherein the selective olefin absorption process comprises using a solvent selected from the group consisting of NMP(n-methyl-2-pyrrolidone), DMF (dimethylformamide), THF (tetrahydrofuran), ACN (acetonitrile), and mixtures thereof An embodiment of the invention is one, any or all of prior embodiments in this paragraph up through the first embodiment in this paragraph further comprising passing the sixth stream comprising paraffins to the dehydrogenation reactor. An embodiment of the invention is one, any or all of prior embodiments in this paragraph up through the first embodiment in this paragraph wherein the second stream is cooled to condense water, compressed and cooled to condense more water before passing the second stream to the drying unit. An embodiment of the invention is one, any or all of prior embodiments in this paragraph up through the first embodiment in this paragraph further comprising passing a portion of the fourth stream comprising lighter gases to the dehydrogenation reactor. An embodiment of the invention is one, any or all of prior embodiments in this paragraph up through the first embodiment in this paragraph wherein the cold-box condensing unit is operated at a temperature below −15° C.

A second embodiment of the invention is a process for the production of butadienes comprising passing a first stream comprising n-butane to a dehydrogenation reactor to generate a second stream comprising n-butane and butenes; passing the second stream to a selective olefin absorption process to generate a third stream rich in olefins and fourth stream rich in paraffins and light gases; passing the fourth stream to a drying unit to generate a dried fourth stream; passing the dried fourth stream to a cold-box separation unit to generate a fifth stream comprising light gases and a sixth stream comprising n-butane; and passing the third stream to an oxydehydrogenation reactor to generate a product stream comprising butadienes. An embodiment of the invention is one, any or all of prior embodiments in this paragraph up through the second embodiment in this paragraph further comprising passing the second stream to a chloride treater to generate a treated second stream An embodiment of the invention is one, any or all of prior embodiments in this paragraph up through the second embodiment in this paragraph wherein the third stream is passed to a solvent separation unit to generate a solvent separation bottoms stream comprising solvent, and a solvent separation overhead stream comprising olefins; and passing the solvent separation overhead stream to the oxydehydrogenation reactor. An embodiment of the invention is one, any or all of prior embodiments in this paragraph up through the second embodiment in this paragraph further comprising passing the sixth stream to the dehydrogenation reactor. An embodiment of the invention is one, any or all of prior embodiments in this paragraph up through the second embodiment in this paragraph further comprising passing a portion of the fifth stream to the dehydrogenation reactor. An embodiment of the invention is one, any or all of prior embodiments in this paragraph up through the second embodiment in this paragraph wherein the cold-box condensing unit is operated at a temperature below −15° C.

A third embodiment of the invention is a process for the dehydrogenation of n-butane comprising passing a first stream comprising n-butane to a dehydrogenation reactor to generate a second stream comprising n-butane and butenes; passing the second stream to first stripping unit to generate a stripped second stream comprising C4 hydrocarbons and a third stream comprising lighter gases; passing the stripped second stream to an extractive distillation zone to generate a fourth stream rich in olefins and a fifth stream rich in paraffins; and passing the fourth stream to an oxydehydrogenation reactor to generate a product stream comprising butadienes. An embodiment of the invention is one, any or all of prior embodiments in this paragraph up through the third embodiment in this paragraph further comprising passing the fifth stream to the dehydrogenation reactor. An embodiment of the invention is one, any or all of prior embodiments in this paragraph up through the third embodiment in this paragraph further comprising passing a portion of the third stream to the dehydrogenation reactor. An embodiment of the invention is one, any or all of prior embodiments in this paragraph up through the third embodiment in this paragraph further comprising passing the second stream to condenser to create a cooled second stream; passing the cooled second stream to a knock-out drum to remove condensed water to generate a second stream with reduced water content; passing the second stream with reduced water content to a compressor to generate a compressed second stream; passing the compressed second stream to a heat exchanger and second knock-out drum to generate a compressed second stream with reduced water content; passing the compressed second stream with reduced water content to a chloride treater to generate a dried and treated second stream; and passing the dried and treated second stream to the first stripping unit. The process of 16 wherein the first stripping unit is an absorber-stripper, and uses a non-specific light oil for absorbing the C4 compounds from the second stream.

Other objects, advantages and applications of the present invention will become apparent to those skilled in the art from the following detailed description and drawings.

### BRIEF DESCRIPTION OF THE DRAWING

FIG. 1 is a first embodiment of a process for producing butadienes with the use of oxydehydrogenation following dehydrogenation of butane;

FIG. 2 is a second embodiment of embodiment of a process for producing butadienes with the use of oxydehydrogenation following dehydrogenation of butane; and

FIG. 3 is a third embodiment of a process for producing butadienes with the use of oxydehydrogenation following dehydrogenation of butane.

### DETAILED DESCRIPTION

The production of 1,3 butadiene in increasing in importance as demand for butyl rubbers and other polymeric materials increases. Currently the primary source of butadiene is through a by-product stream from cracking processes in the production of light olefins. The present process is for an on-purpose production of butadiene from a butane rich feedstream. The economics of a process is dependent on the purity of the butene feed used, with lower purity feeds, i.e. streams with lower butene content, having a negative impact on the process. This is due to the increase in volume of reactants sent to the reactors, as well as the lower concentration of butadienes generated and lower concentration of butadiene in the product stream.

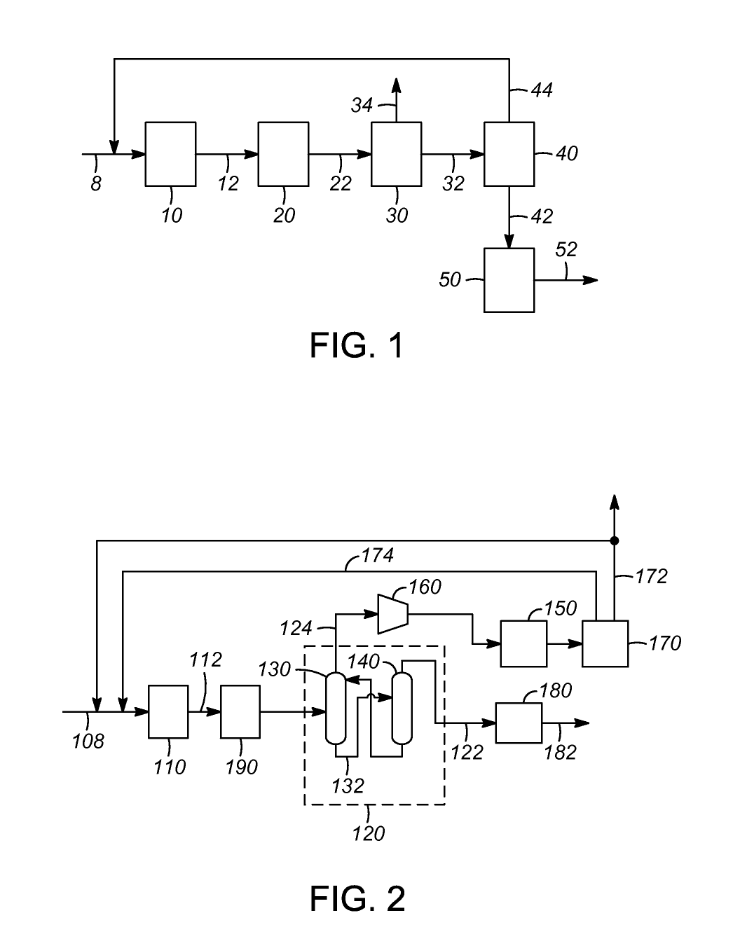

One aspect of this process is to convert n-butane to butenes, and subsequently sending the butene process stream to an oxidative dehydrogenation zone to complete the butadiene production. One embodiment of the present invention can be seen in FIG. 1.

The process includes passing a first stream 8 having n-butane to a dehydrogenation reactor 10 to generate a second stream 12 comprising n-butane and butenes. The second stream 12 is passed to a drying unit 20 to generate a dried stream 22. The dried stream 22 is passed to a cold-box condensing unit 30 to generate third stream 32 comprising liquid C4 hydrocarbons and a fourth stream 34 comprising lighter gases, including hydrogen. The third stream 32 is passed to an olefin separation zone 40 to generate a fifth stream 42 rich in olefins and a sixth stream 44 rich in paraffins. The fifth stream 42 is passed to an oxydehydrogation reactor 50 to generate a product stream 52 comprising butadienes. The butadienes in the product stream 52 comprise 1,3 butadiene which can be separated and recovered with other C4 compounds recycled, or processed elsewhere. The sixth stream 44 is passed to the dehydrogenation reactor 10. A portion of the fourth stream 34 is passed to the dehydrogenation reactor 10.

As used herein, the term reactor can refer to either a singular reactor or a plurality of reactors that are performing the same function. The reactor can also comprise a plurality of reaction beds, and can be moving or fixed bed reactors, although moving bed reactors with a regeneration system are preferred.

The process can further include a chloride treatment of the process streams. The chloride treatment can be a part of the drying unit 20, or can be an additional unit that is added at the end of the drying unit 20. The dried stream 22 is passed through a chloride treater to remove residual chloride that can be generated as a result of the dehydrogenation reactor, where residual chloride results from the regeneration process for the catalyst.

The drying unit 20 can comprise a system of condensers and knockout drums to remove water, with a compressor and condenser to further knockout moisture, as well as a final drying bed of adsorbent for removing residual water from the process stream. The drying unit 20 removes moisture to prevent problems in the cold-box condensing unit 30.

Butanes and butenes have moderate boiling points that are near the freezing point of water. Cooling a gas stream comprising butanes and butenes can be accomplished with a low expense cold-box technology. The gas can be first expanded to cool the gas, then passed through a heat exchanger that cools the expanded gas to a temperature below −15C. The C4 hydrocarbons then condense while lighter gases such as hydrogen and any carbon oxides remain in the gaseous phase. The cold-box condensing unit 30 is operated at a temperature to sufficiently condense the C4 hydrocarbons. Different C4 hydrocarbons condense at temperatures between 1° C. and −12° C. at one atmosphere. The cold-box unit 30 is preferably operated at a temperature below −15° C.

The olefin separation zone 40 comprises a solvent separation for separation of olefins from paraffins. An appropriate solvent is a solvent comprising a polar nitrogen compound, or a mixture of polar compounds. Examples of solvents, though not limited to these, include n-methylpyrrolidone (NMP), dimethylformamide (DMF), dimethyl acetamide, and acetonitrile (ACN). A common extractive solvent is NMP. The olefin separation zone 40 can include at least one fractionation unit for the recovery of solvent and recycling of the solvent to the solvent separation zone within the olefin separation zone 40.

Another embodiment of the present invention is presented in FIG. 2. The process involves passing a first stream 108 having n-butane to a dehydrogenation reactor 110 to generate a second stream 112 having n-butane and butenes. The second stream 112 is passed to a selective olefin absorption unit 120 to generate a third stream 122 rich in olefins, and a fourth stream 124 having light gases and paraffins. The fourth stream 124 is passed to a drying unit 150 to generate a dried fourth stream 152. The fourth stream can also be passed through a compressor 160 before passing the stream to the drying unit 150. The dried fourth stream 152 is passed to a cold-box separation unit 170 to generate a fifth stream comprising light gases 172 and a sixth stream 174 comprising n-butane. The third stream 122 is passed to an oxydehydrogenation reactor 180 to generate a product stream 182 comprising butadienes.

The selective olefin absorption unit 120 comprises using an absorption unit 130 to separate the olefins and paraffins. The olefins and a solvent are passed in a stream 132 to a solvent separation unit 140 to generate an overhead stream, or the third stream 122 rich in olefins and a bottoms stream, or a solvent stream 142 for recycle to the absorption unit 130. The paraffins are passed out in the fourth stream 124.

The process can further include passing the second stream 112 to a pretreatment unit 190 before passing the second stream 112 to the selective solvent absorption unit 120. The pretreatment unit 190 can include a chloride treater to generate a treated second stream. The chloride treater removes chloride that is given off the catalyst during the dehydrogenation reaction as the catalyst ages. The pretreatment unit 190 can include condensers and compressors for cooling and compressing the second stream 112, and for removing some of the moisture generated in the dehydrogenation reactor 110.

The process can further include passing the sixth stream 174 to the dehydrogenation reactor 110, and a portion of the fifth stream 172 having hydrogen can be passed to the dehydrogenation reactor 110.

One option with the second embodiment, is to use and absorber/stripper system in place of the cold-box condensing unit 170, wherein the absorber/stripper separates the light gases including hydrogen from the paraffins.

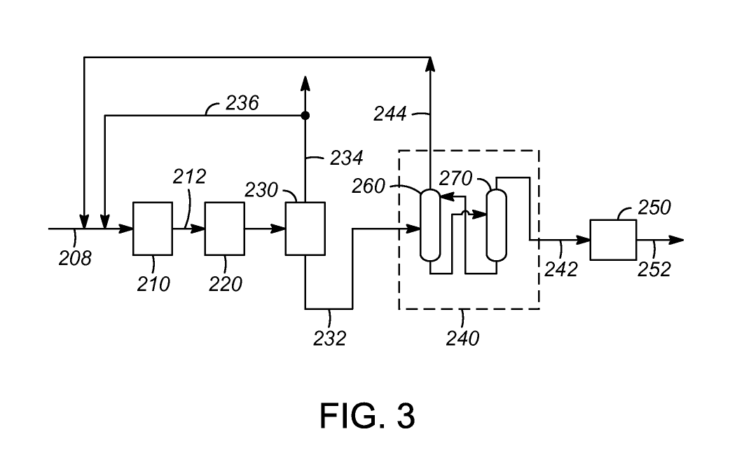

In a third embodiment, the process is presented in FIG. 3. The process includes passing a first stream 208 having n-butane to a dehydrogenation reactor 210. The dehydrogenation reactor 210 converts the n-butane to butenes and generates a second process stream 212. The second process stream 212 is passed to a first stripping unit 230 and generates a stripped second stream 232 and a third stream 234. The stripped second stream 232 includes C4 hydrocarbons, and the third stream 234 includes lighter gases. The stripped second stream 232 is passed to an extractive distillation zone 240 to separate the olefins from the paraffins. The extractive distillation zone 240 generates a fourth stream 242 rich in olefins, and a fifth stream 244 rich in paraffins. The fourth stream 242 is passed to an oxydehydrogenation reactor 250 to generate a product stream 252 having butadienes. The fifth stream 244 can be passed to the dehydrogenation reactor 210, and a portion 236 of the third stream 234 having hydrogen can be passed to the dehydrogenation reactor 210.

In one aspect of the third embodiment, the first stripping unit 230 is an absorber-stripper, and uses a non-specific light oil for absorbing the C4 compounds from the stripped second stream. A non-specific light oil can comprise a non-polar hydrocarbon light oil.

The second process stream 212 can be passed to a cooling and condensing system 220 before passing the second process stream to the first stripping unit 230. The cooling and condensing system includes passing the second stream 212 to a condenser to create a cooled second stream. The cooled second stream passed to a knock-out drum to remove condensed water and to knock out liquids from the cooled stream. The vapor stream is passed to a compressor to generate a compressed second stream. The compressed second stream is further cooled through a condenser to condense more water from the second stream. The compressed second stream is then passed to a knock-out drum to separate liquid from vapor to generate a reduced moisture second stream. The reduced moisture second stream is then passed to a drying unit and a chloride treater to remove residual moisture and any chloride leached from the dehydrogenation reactor catalyst. The dried and treated second stream is then passed to the first stripping unit 230.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

What is claimed is:

1. A process for the dehydrogenation of n-butane comprising:
passing a first stream comprising n-butane to a dehydrogenation reactor to generate a second stream comprising n-butane and butenes;
passing the second stream to a drying unit to generate a dried second stream passing the dried second stream to a cold-box condensing unit to generate a third stream comprising liquid C4 hydrocarbons and a fourth stream comprising lighter gases;
passing the third stream to an olefin separation process to generate a fifth stream rich in olefins and a sixth stream rich in paraffins; and
passing the fifth stream to an oxydehydrogenation reactor to generate a product stream comprising butadienes.

2. The process of claim 1 wherein the butadienes in the product stream comprising 1,3 butadiene.

3. The process of claim 1 further comprising passing the second stream with reduced water content to a chloride treater to generate a treated second stream.

4. The process of claim 1 further wherein the olefin separation comprises:
passing the third stream to a selective olefin absorption process to generate the fifth stream comprising olefins and the sixth stream comprising paraffins.

5. The process of claim 4 wherein the selective olefin absorption process comprises using a solvent selected from the group consisting of NMP(n-methyl-2-pyrrolidone), DMF (dimethylformamide), THF (tetrahydrofuran), ACN (acetonitrile), and mixtures thereof.

6. The process of claim 1 further comprising passing the sixth stream comprising paraffins to the dehydrogenation reactor.

7. The process of claim 1 wherein the second stream is cooled to condense water, compressed and cooled to condense more water before passing the second stream to the drying unit.

8. The process of claim 1 further comprising passing a portion of the fourth stream comprising lighter gases to the dehydrogenation reactor.

9. The process of claim 1 wherein the cold-box condensing unit is operated at a temperature below −15° C.

10. A process for the production of butadienes comprising:
passing a first stream comprising n-butane to a dehydrogenation reactor to generate a second stream comprising n-butane and butenes;
passing the second stream to a selective olefin absorption process to generate a third stream rich in olefins and fourth stream rich in paraffins and light gases;
passing the fourth stream to a drying unit to generate a dried fourth stream;
passing the dried fourth stream to a cold-box separation unit to generate a fifth stream comprising light gases and a sixth stream comprising n-butane; and
passing the third stream to an oxydehydrogenation reactor to generate a product stream comprising butadienes.

11. The process of claim 10 further comprising passing the second stream to a chloride treater to generate a treated second stream.

12. The process of claim 10 wherein the third stream is passed to a solvent separation unit to generate a solvent separation bottoms stream comprising solvent, and a solvent separation overhead stream comprising olefins; and
passing the solvent separation overhead stream to the oxydehydrogenation reactor.

13. The process of claim 10 further comprising passing the sixth stream to the dehydrogenation reactor.

14. The process of claim 10 further comprising passing a portion of the fifth stream to the dehydrogenation reactor.

15. The process of claim 10 wherein the cold-box condensing unit is operated at a temperature below −15° C.

16. A process for the dehydrogenation of n-butane comprising:
passing a first stream comprising n-butane to a dehydrogenation reactor to generate a second stream comprising n-butane and butenes;
passing the second stream to first stripping unit to generate a stripped second stream comprising C4 hydrocarbons and a third stream comprising lighter gases;
passing the stripped second stream to an extractive distillation zone to generate a fourth stream rich in olefins and a fifth stream rich in paraffins; and
passing the fourth stream to an oxydehydrogenation reactor to generate a product stream comprising butadienes.

17. The process of claim 16 further comprising passing the fifth stream to the dehydrogenation reactor.

18. The process of claim 16 further comprising passing a portion of the third stream to the dehydrogenation reactor.

19. The process of claim 16 further comprising:
passing the second stream to condenser to create a cooled second stream;
passing the cooled second stream to a knock-out drum to remove condensed water to generate a second stream with reduced water content;
passing the second stream with reduced water content to a compressor to generate a compressed second stream;
passing the compressed second stream to a heat exchanger and second knock-out drum to generate a compressed second stream with reduced water content;
passing the compressed second stream with reduced water content to a chloride treater to generate a dried and treated second stream; and
passing the dried and treated second stream to the first stripping unit.

20. The process of 16 wherein the first stripping unit is an absorber-stripper, and uses a non-specific light oil for absorbing the C4 compounds from the second stream.
